(19) United States
(12) Patent Application Publication
(10) Pub. No.: US 2018/0258142 A1
(43) Pub. Date: Sep. 13, 2018

(54) Methods for Optimizing CATS Immunotherapeutics into Humanized Derivatives with Reduced Autoreactivity, Reduced Toxicity and Enhanced Long-Term Efficacy

(71) Applicant: Cherry Med, Inc., Sugar Land, TX (US)

(72) Inventor: David I. Cohen, Pelham, NY (US)

(21) Appl. No.: 15/917,761

(22) Filed: Mar. 11, 2018

Related U.S. Application Data
(60) Provisional application No. 62/469,702, filed on Mar. 10, 2017.

(51) Int. Cl. C07K 14/005; C07K 16/28; C07K 16/22; C07K 16/46; A61P 35/00; A61P 35/04; C07K 14/715; C07K 14/82

(52) U.S. Cl. CPC C07K 14/005; C07K 2319/50; C07K 16/22; C07K 16/468; A61P 35/00; A61P 35/04; C07K 14/7151; C07K 14/82; C07K 2317/31; C07K 2317/35; C07K 2317/24; C12N 2740/16022; C12N 2740/16033; C07K 2319/00; C07K 16/2878

(57) ABSTRACT

The present invention describes unique means for reducing autoreactivity that sensitizes against sustained treatments, and toxicity associated with administration of biologicals, in order to develop safer cancer immunotherapeutics. Short functional sequences are identified in the biologic and matched to their most homologous human counterpart. The human homologs are swapped in to replace their foreign counterparts. Alternatively, variants are selected that have exhibit less toxicity in human or primate experiments from nature, and these sequences are swapped in to replace their counterparts in the biologic. Also, human sequences that mediate the same function as foreign sequences in the biologic, but lack any sequence homology, can be swapped in for their foreign sequence counterparts. These inventions are applied to derivatize the HIV/SIV Tat protein into humanized trimers (CATS) useful for the treatment of cancer.

### FIELD OF THE INVENTION

The present invention relates to the field of immune-based therapeutic agents in the treatment of cancer at every stage of disease, and their use in combination to achieve long term remissions and curative activity.

### BACKGROUND

Cancer is among the leading causes of morbidity and mortality worldwide with approximately 14 million new cases and 8.2 million cancer related deaths in 2012 (WHO, World Cancer Report. Bernard W. Stewart and Christopher P. Wild, eds. 2014). The number of new cases is expected to rise by about 75% over the next 2 decades coincident with an aging population. One defining feature of cancer is the rapid creation of abnormal cells that grow beyond their usual boundaries, and which can then invade adjoining parts of the body and spread to other organs. The transformation from normal cells into tumor cells is a multistage process, typically a progression from pre-cancerous lesions seeded by cancer stem cells, to malignant tumors that metastasize to distant sites. Metastasis is the primary cause of death for human cancers, while certain cancers that rarely metastasize (basal cell carcinoma) are almost never fatal. Oncogenesis is the result of the interaction between genetic factors and external agents such as, but not limited to, ultraviolet radiation, asbestos tobacco smoke, or viral infection.

Breast and prostate cancers are among the most frequently diagnosed malignancies in the United States other than skin cancers. Generally, these cancers can only be curatively resected, when detected early, and resection has little if any role in metastatic cancer. Non-surgical approaches, such as radiotherapy or chemotherapy, affect normal cells and result in side effects that limit treatment. Importantly, all current treatments for recurrent or metastatic cancer are only palliative. Consequently, development of novel systemic approaches to treat advanced, recurrent and metastatic cancers are urgently needed, particularly insofar as these approaches offer an extended quality of life to the diseased individual.

The selectivity and safety of cancer treatment with biologics can be improved by improving deliverability designed directly at growing cancer cells, and by reducing toxicities that are not required for cancer cell killing. Such toxicities include bystander normal cell death, and auto-reaction directed at the biologics, as well as toxicities associated directly with the particular biologic.

Immunotherapy has great promise as a treatment for cancer patients because of its specificity and freedom from many of the toxic effects of chemotherapies. Cancer is one of many common human diseases that respond to immune-based treatments. Clinical trials in humans have established that an immune response could regress some human melanomas, prostate cancers, renal cancers, and lung cancers. Immunotherapy for cancers constitute a broad range of biologics including monoclonal antibodies, human cytokines such as GM-CSF, cancer vaccines wherein cancer antigens are delivered along with an innate immune stimulant. Each of these technologies is associated with some limitation, but all require that the biologic bear maximal homology to human sequences in order to minimize autoreactive side effects. Since cancer immunotherapeutics attempt to stimulate an immune response, as opposed to, for example immunotherapeutics to autoimmune diseases (Humira® Abbott Biotechnology Ltd., Remicade® Centocor, Inc.) there is an even more pressing need to match the immunotherapeutic to human sequences that are well-tolerated because not recognized by the immune system as foreign substances.

With the advent of monoclonal antibody technology, researchers and clinicians had access to essentially unlimited quantities of uniform antibodies capable of binding to a predetermined antigenic site and having various immunological effector functions. Unfortunately, the development of appropriate therapeutic products based on these proteins has been hampered by a number of drawbacks inherent in monoclonal antibody production. For example, most monoclonal antibodies are mouse derived, and thus do not fix human complement well. They also lack other important immunoglobulin functional characteristics when used in humans. Perhaps most importantly, non-human monoclonal antibodies contain amino acid sequences that will be immunogenic when injected into a human patient. Numerous studies have shown that after injection of a foreign antibody, the immune response mounted by a patient can be quite strong, essentially eliminating the antibody's therapeutic utility after an initial treatment. With the use of different mouse monoclonal antibodies to treat various diseases and after one or several treatments with non-human antibodies, subsequent treatments, even for unrelated therapies, can be ineffective or even dangerous in themselves, because of cross-reactivity.

Even with chimeric antibodies, where the mouse variable regions are joined to human constant regions, a significant immunogenicity problem remains. Moreover, efforts to immortalize human B-cells or generate human hybridomas capable of producing human immunoglobulins against a desired antigen have been generally unsuccessful, particularly with many important human antigens. More recently, human framework regions combined with complementarity determining regions (CDR's) from a donor mouse immunoglobulin (see, EPO Publication No. 0239400) have been described as “humanized” immunoglobulins and the process by which the donor immunoglobulin is converted into a human-like immunoglobulin by combining its CDR's with a human framework is called “humanization”. Humanized antibodies are important because they bind to the same antigen as the original antibodies, but are less immunogenic when injected into humans.

However, one problem with present humanization procedures has been a loss of affinity for the antigen, meaning that more of the humanized antibody will have to be injected into the patient, at higher cost and greater risk of adverse effects. Further while “humanization” of monoclonal antibody (MAb) therapeutics is now standard, the problem of autoreactive toxicity in cancer immunotherapeutics is compounded by their counter suppressive nature, as evidenced by severe autoimmune reactions (“cytokine storms”) in recent cancer clinical trials (Yervoy® Bristol-Myers Squibb Company).

Thus, there is a need for an improved means to develop humanized biologic therapeutics for cancer. Ideally, these humanized biologics should remain substantially non-immunogenic in humans, yet be easily and economically produced in a manner suitable for therapeutic formulation and other uses. The present invention provides a means both to develop a humanized version of trimeric HIV TAT as immune therapeutic for cancer, and to produce the therapeutic more efficiently. The humanization strategy utilizes a better understanding of the component activities of short genetic sequences (tiles) that are stitched together to comprise the anti-cancer functions of trimeric TAT. Based upon an absolute requirement for a C-rich determinant (CRD) unusual both for its length and apparent chimeric construction, such cancer or CRD adjuvant therapeutics (CATS) can be humanized empirically with variant CRD sequences consistently associated with reduced neurotoxicity and other toxicity, identified from the literally thousands of HIV or SIV TAT isolates. Two such examples are provided here. The CRD of TAT/CATS is analogous to the CDR of a monoclonal antibody insofar as both are their “active sites,” but despite the similarity in their abbreviations they bear no sequence homology to one another.

Alternatively, a CATS CRD can be comprised of short sequences associated with function that are matched to their most conserved human counterparts for these short functional sequences. The successful empirical trial of such a “fully humanized” CATS CRD would open the door for genetic constructs comprised of CRD not anticipated by nature that could trigger novel therapeutic activities. Furthermore, matching of human genetic components to TAT's

CRD would support a program to make therapeutic MAb to these components, as a second way to harness trimeric TAT immuotherapeutic (TIRX) potential. Of course such MAb therapeutics would require their own humanization by strategies now common to the industry.

Other viral sequences in TIRX can be replaced by human sequences mediating the same function. As one such example, TIRX trimerization sequence, identified in the figures, can be replaced by the trimer-forming leucine zipper (aa 111-138) of human tenascin precursor (SEQ ID NO. 2), or by the trimer-forming isoleucine zipper of TNF-related apoptosis-inducing ligand (TRAIL: SEQ ID NO. 3). Zipper domains facilitate stable re-annealing of trimer from monomer, which could improve CATS production in insect cells where the trimer forms spontaneously but is relatively unstable, and support active CATS production in E.coli where re-annealing of trimer from monomer is requisite. Detailed description of this methodology with E.coli GCN4 (SEQ ID NO. 5) is available (Protein Engineering, Design & Selection vol. 21 no. 1 pp. 11-18, 2008). While this latter strategy could facilitate production, it would not humanize the resultant compound. Human equivalents to E.coli GCN4 (SEQ ID No. 5) have been described, e.g. human GITRL (SEQ ID NO. 7) and ATF-4 (SEQ ID NO. 6).

A third methodology for reducing toxicity is to mutate and thereby cripple domain(s) implicated by experiments of nature in toxicity. The amino terminal SH3-binding (SH3B) sequence of TAT, identified in the figures, is such a domain. Its loss of function construct can be engineered by replacing internal proline residues with valine. Such a construct maintains CATS immunotherapeutic function as shown in the figures.

### SUMMARY

By anticipating and eliminating auto-reactivity that sensitizes against sustained treatments, the present invention discloses a system for perfecting efficacy of CATS biologics. Additional strategies are described that either inactivate toxic viral sequences, or replace viral sequences with functionally equivalent human sequences. In the case of sequences that mediate the trimerization of TAT, CATS can be formulated with human sequences that generate a tighter and more efficiently formed trimer. Further, in order to develop safer cancer immunotherapeutics, short and inexpensive steps are taken in the clinical development of biologics through small animal cancer models, human models of neurotoxicity, and other cancer trials in animals. By screening and reducing auto-reactivity in each step of development, potential problems are addressed earlier rather than at more expensive late stages.

### DESCRIPTION OF THE FIGURES

FIG. 1. Schematic Depicting five Tiles Of HIV TAT (SEQ ID NO. 1) and their derivatization into a Humanized CATS Immunotherapeutic with Enhanced Stability.

From its amino terminus (aa 1-19) Tat encodes a transduction peptide, either an acidic activator or SH3 binding domain (SH3B), followed by a CRD with immunomodulatory ligand activity (aa20-38), followed by the well known TAR-MTS sequence (39-57), followed by a loop with acid protease cleavage sites (58-72), ending with a divalent-cation dependent trimerization sequence. These sequences can be modified individually or in groups to create “humanized” CATS that are better tolerated and more efficacious than TIRX. Position designations refer to HIV-1 TAT (SEQ ID NO. 1).

FIG. 2. Graphic depicting the CRD region Of TAT.

Above. Cn3D view, CRD highlighted in light yellow. Below. Linear schematic, CRD light blue.

FIG. 3. CRD as the Active site of CATS.

BALB/c mice implanted with 1×10⁴ 4T1 tumor cells were treated s.c. on days 0, day 7, day 14 and day 21with (A) CATS derivatives (400ng) or (B) a TAT derivative with βdefensin 4 (SEQ ID NO. 9) swapped to replace TAT CRD (SEQ ID NO. 1) (400ng and 2ug s.c.); the control group was treated with PBS. At either dose a βdefensin 4 CRD (SEQ ID NO. 9) is completely inactive against 4T1 breast cancer.

FIG. 4. Homology between TAT CRD (SEQ ID NO. 1) and the WNT superfamily of differentiation proteins. (SEQ ID NOS. 10, 11, 11, 12, 13, 14) Alignment structured with the MultAlin tool (INRA).

FIG. 5. Homology between TAT CRD (SEQ ID NO. 1) and TNF receptor 1 (SEQ ID NO. 15).

Illustrated is the exact match of the dual C-rich regions spanning respectively only 5 or 6 amino acids when TAT is compared against bat (SEQ ID NO. 15) as detected by the BlastP program.

FIG. 6. Comparative anti-tumor activity of TIRX versus a prototype “humanized” CATS in the TS/A breast cancer model.

Mice (ten per cohort) were implanted s.c. on Day 0 with 1×10⁵ TS/A breast cancer cells and treated weekly starting on Day 1 with 10 ng IV inactive protein (Control, Blue), TIRX (Magenta) or CATS (Green). 1⁰ tumor volume is recorded. The difference in 1⁰ growth suppression by CATS over TIRX is highly statistically significant (P <0.01).

FIG. 7. Graph Showing Tumor Immunomodulatory Activity Of CATS Resident In Trimer.

Trimeric Tat immunotherapeutics (TIRX) but not monomer Tat suppress 1⁰ tumor growth of 4T1 murine breast cancer at ng dosing in vivo. A. Western blot of recombinant Tat proteins synthesized in baculovirus after incubation in 10 mM EDTA (Lane 1), 1 mM EDTA (Lane 2),or as directly isolated and purified from insect cells (Lanes 3) probed with polyclonal antibodies to HIV1 SF2 peptide. B. 30 BALB/c mice were implanted in the mammary pad on Day 0 with 1×10⁴ 4T1 cells. Beginning on Day 1 and weekly thereafter, ten mice per cohort were treated with either trimcric TAT (TIRX 10 ng IV, purple) which multimerizes spontaneously in baculovirus, standard of care cyclophosphamide (CY 80mg/kg intraperitoneally (IP), green), or monomeric Tat (M-Tat 10 ng IV, brown) produced by incubating TIRX in 10 mM EDTA. Data represent mean tumor volume as calculated (length (mm)×width(mm)²)×0.52); bars, ±SE. The difference between CY vs TIRX treatment groups is very highly statistically significant (p<0.005).

FIG. 8. Schematic Of The Loop And Tail Regions Of Tat.

Above. Left. Loop region, highlight red. Right. Carboxyl tail, highlighted green. Cn3D model. Below. Linear representation of the loop (red) and tail (blue).

FIG. 9. Tail Amino acids (66-101) of TAT mediate spontaneous trimerization in Insect Cells. Baculovirus constructs expressing full length, 2 exon TAT, or a construct truncated after aa 65 were used to infect Sf9 insect cells. Proteins were harvested and resolved by SDS-PAGE gel electrophoresis, and probed by immunoblot with polyclonal anti-TAT antibodies. Trimeric TAT resolves at ˜45 kd, and monomeric TAT runs as a 16 kD protein.

FIG. 10. SH3 Binding Domain in Pathogenic HIV-1 TAT (SEQ ID NO. 1) has homologies to WNT-1 (SEQ ID NOS. 10, 11, 11, 12, 13, 14).

A. Cn3D depiction (yellow) and block figure (light blue) of NH₂ signal transduction peptide (STP). B. Praline alignment of HIV-1 (SEQ ID NO. 1), HIV-2 (SEQ ID NO. 16), and CPZ TAT (SEQ ID NO. 17) have perfect alignment of proline residues, while other intervening sequences are more variable. C. MultAlin of HIV-1 TAT (SEQ ID NO. 1) and WNT-1 (SEQ ID NO. 10) demonstrates alignment with two deletions in the viral STP.

FIG. 11. Block Depiction of a fully-humanized CATS.

Some or all of these components can comprise any individual CATS compound designed for enhanced safety, production, stability, and efficacy.

### DETAILED DESCRIPTION OF THE INVENTION

Immunotherapy is a targeted approach that could control tumor growth and prevent metastases while avoiding many of the side effects associated with standard chemotherapy. This is particularly relevant in breast cancer where early breast cancer immunotherapy has focused on targeting the immune response against cancer cells with the administration of a vaccine or monoclonal antibody for a breast cancer antigen.

MAb “humanization” for therapeutic use is routinely performed prior to clinical trials. Unfortunately the problem of autoreactive toxicity to cancer immunotherapeutics that further stimulate any immune response has not been adequately addressed in the prior art. Their risk of triggering severe autoimmune reactions such as “cytokine storms” has been reported in several recent cancer clinical trials. The method of the present invention provides a simple platform for mitigating risks at later stage clinical trial. The innovation of this program is to use the humanization steps routinely used in MAb development, in the development of biologic oncoimmunologic (O-I) agonist. The present invention describes a biologic designed for reduced auto-reactivity that can translate safely and efficaciously from animal trials into human clinical trial.

One embodiment of the present invention anticipates and eliminates auto-reactivity that sensitizes against sustained treatments. This strategy aims to avert failure at costly late-stage clinical trials. For example, Dynavax' TLR adjuvants for hepatitis B vaccines, tested initially in mice, were given to humans at higher doses (Jason D. Marshall, Edith M. Hessel, Josh Gregorio, Christina Abbate, Priscilla Yee, Mabel Chu, Gary Van Nest, Robert L. Coffman, and Karen L. Fearon. Novel chimeric immunomodulatory compounds containing short CpG oligodeoxyribonucleotides have differential activities in human cells. Nucleic Acids Res. 31: 5122-5133, 2003). Dynavax Phase III trial experienced a costly hold when a vaccine developed Wegener's granulomatosis presumed to be a reaction against adjuvant (Hurtado PR, Jeffs L, Nitschke J, Patel M, Sarvestani G, Cassidy J, Hissaria P, Gillis D, Peh CA. CpG oligodeoxynucleotide stimulates production of anti-neutrophil cytoplasmic antibodies in ANCA associated vasculitis. BMC Immunol. 9:34, 2008). While “humanization” of monoclonal antibody (MAb) therapeutics (Maher VE, Drukman Si, Kinders RJ, Hunter RE, Jennings J, Brigham C,

Stevens S, Griffin TW. Human antibody response to the intravenous and intraperitoneal administration of the F(ab′)2 fragment of the OC125 murine monoclonal antibody. J Immunother 1:56-66, 1992) is now standard, the problem of autoreactive toxicity in cancer immunotherapeutics is compounded by their counter suppressive nature, as evidenced by severe autoimmune reactions (“cytokine storms”) in recent cancer clinical trials (Morgan RA, Yang JC, Kitano M, Dudley ME, Laurencot CM, Rosenberg SA. Case report of a serious adverse event following the administration of T cells transduced with a chimeric antigen receptor recognizing ERBB2. Mol Ther. 18:843-851,2010; Voskens CJ, Goldinger SM, Loquai C, Robert C, Kaehler KC, Berking C, Bergmann T, Bockmeyer CL, Eigentler T, Fluck M, Garbe C, Gutzmer R, Grabbe S, Hauschild A, Hein R, Hundorfean G, Justich A, Keller U, Klein C, Mateus C, Mohr P, Paetzold S, Satzger I, Schadendorf

D, Schlaeppi M, Schuler G, Schuler-Thurner B, Trefzer U, Ulrich J, Vaubel J, von Moos R, Weder P, Wilhelm T, Goppner D, Dummer R, Heinzerling LM. The price of tumor control: an analysis of rare side effects of anti-CTLA-4 therapy in metastatic melanoma from the ipilimumab network. PLoS One. 8:e53745, 2013).

One embodiment of the present invention converts a novel C-Rich domain (CRD) that functions as a ligand-like immunomodulator for all stages of murine breast cancer into a safe and sustained therapy for recurrent breast cancers. This is accomplished by swapping in natural variants of the TAT CRD with either 6 C residues (SEQ ID NO. 18), as has been described in Indian HIV Clade C variants with little neurotoxicity (Ranga et. al. JOURNAL OF VIROLOGY, March 2004, p. 2586-2590) or with 8 C residues (SEQ ID NO. 19), as described in SIV-AGM3 which has minimal immunopathology or neurotoxicity (Broussard et. al., Simian Immunodeficiency Virus Replicates to High Levels in Naturally Infected African Green Monkeys without Inducing Immunologic or Neurologic Disease, J. Virol. 2001, 75:2262.) These natural variants with low toxicity replace the toxic HIV TAT CRD (SEQ ID NO. 1) containing an odd 7 C with a fully paired (6 or 8) CRD. Cysteine pairing is a conserved property of natural human CRD. Additionally, sequences derived from human TNFR, from human WNT-1, or both, identified by homology search, can be swapped into the CRD and evaluated for efficacy and safety.

One embodiment incorporates trimerization strategies to replace the divalent-cation dependent trimerization sequences of TAT with isoleucine or leucine zippers that are less immunogenic, as derivatized from human sequences, and more stable, as lacking cleavage sites. These strategies make for better stability of the product and higher yields of trimer. Trimeric TAT ImmunoTherapeutics (TIRX) deliver, highly statistically significant (p<0.01) reductions in primary tumor mass (˜50% on average), diminution of pulmonary metastases (approximately 80-90% dependent on study protocols), and survival benefits surpassing by at least two fold paclitaxel standard of care in advanced murine orthotopic 4T1 breast cancer However, TIRX are a potent antigen rapidly inducing self-directed antibodies that neutralize anti-breast cancer activity, limit response time and strength, and occasionally trigger autoreactive toxicities including sudden deaths bearing marked similarities with “cytokine storms” associated with other cancer immunotherapeutics.

One embodiment inactivates WNT-1 like SH3B sequences at the amino terminus of TAT by replacing internal P residues with valine. This embodiment either alone, or in combination with substitutions in the loop region that block the generation of monomer, such as replacement of cleavable S and T residues with A, or substitution of the loop with a polyG spacer, negate activity of the SH3B domain. A preferred embodiment of this invention, described previously for DAGRs, replaces the inhibitory WNT-1 sequences with immunostimulatory sequences from the CRK oncogene (SEQ ID NO. 8).

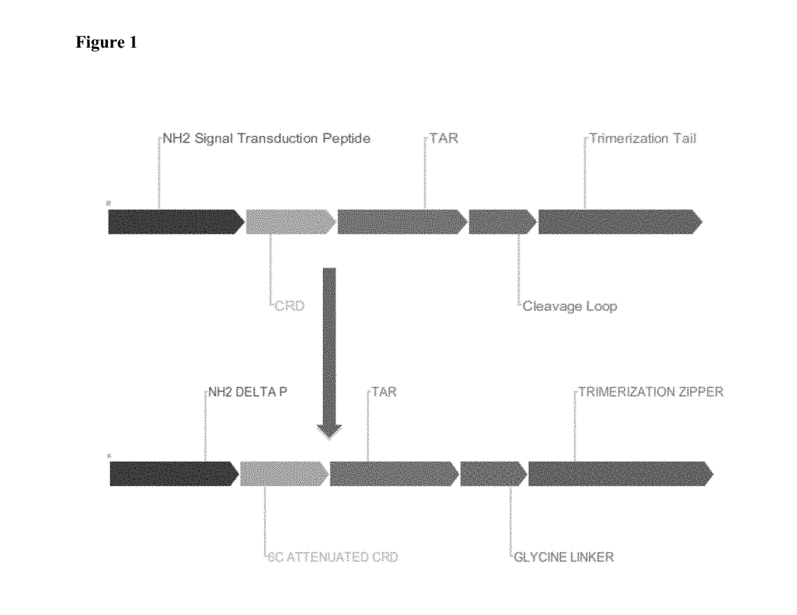

FIG. 1. Schematic Depicting five Tiles of HIV TAT and their derivatization into a Humanized CATS immunotherapeutic with Enhanced Stability.

The native HIV TAT protein (SEQ ID NO. 1) was broken down into 5 distinct activities through extensive homology searches, deep data mining, and functional swap derivatizations. At its amino terminus TAT encodes a transduction peptide, typically an acidic activator. At some point during its evolution SIV incorporated an SH3 binding (SH3B) domain instead of an acidic activator, at the same time it became more virulent. The SH3B is found only in HIV-1 (SEQ ID NO. 1), some HIV-2 (SEQ ID NO. 16), and CPZ (SEQ ID NO. 17), all viruses that promote a rapid course to AIDS. As for any SH3B, this function can be inactivated through the preferred conversion of internal P to V.

TAT's CRD bears strong homology at its amino half to the two C-flanking regions of TNFR1 (SEQ ID NO. 15), and at its COOH half to WNT-1 (SEQ ID NO. 10). Interestingly the C-flanking regions of TAT and bat TNFR1 bear perfect homology. Either or both human peptides can be engineered to replace TAT's CRD, or MAb against the two, including bivalent MAb, could be generated in an attempt to trigger the anti-cancer O-I activity of TAT.

The cleavage loop that facilitates transition of trimeric TAT to monomer TAT can be stabilized with the replacement of each cleavable S or T residue with A. Alternately, it can be removed entirely and replace with a poly G linker in the context of a leucine or isoleucine trimerization domain. This latter strategy has the advantage that a highly immunogenic sequence of TAT, as measured by its mutability, is replaced by a markedly less immunogenic sequence. The second exon of HIV TAT, as demonstrated in FIG. 9, encodes sequences necessary for trimerization. These sequences, like the cleavage loop, are highly immunogenic as demonstrated by mutability from immune pressure. Replacement with a human isoleucine or leucine trimerization domain has the duel advantages of stabilizing the CATS trimer, and of humanizing a previously autoreactive domain.

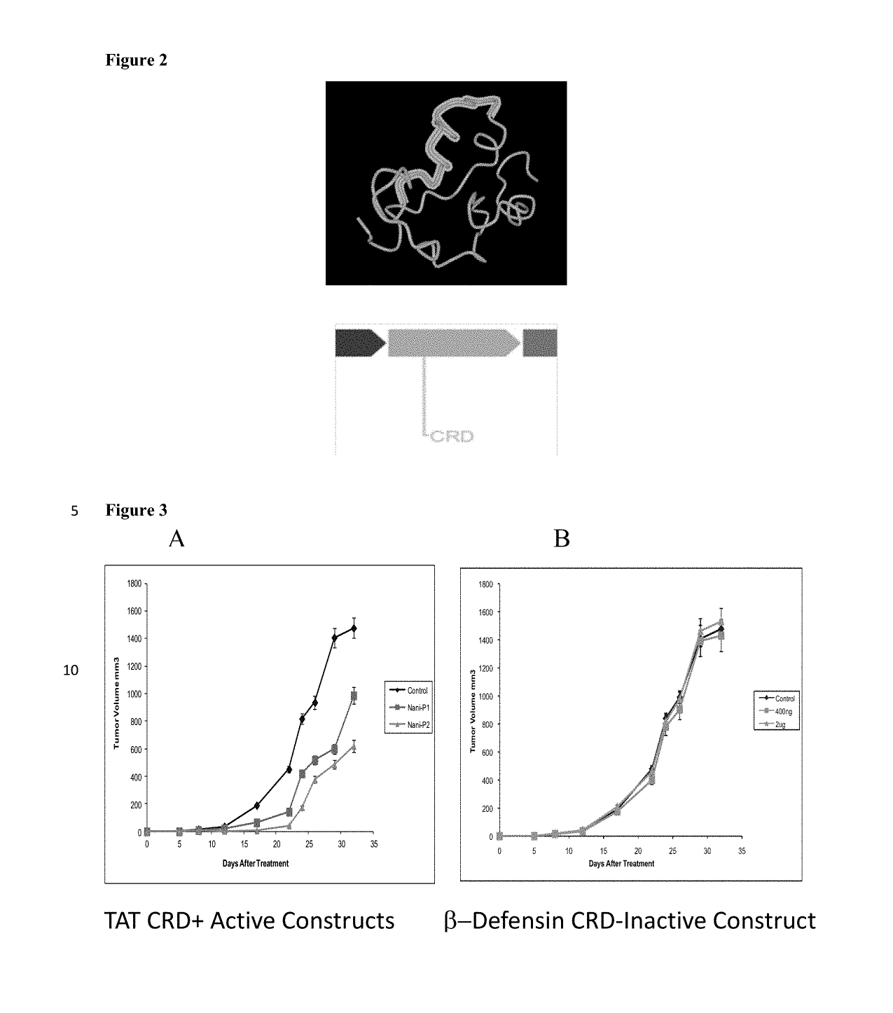

FIG. 2. Graphic depicting the CRD region of TAT.

FIG. 3. CRD as the Active site of CATS.

Comparison between CATS and a TAT derivative bearing a βdefensin 4 CRD (SEQ ID NO. 9). The more active CATS also has a modified SH3B in which internal P are replaced with V. Data represents mean tumor volume calculated as (length (mm)×width (mm)²)×0.52; bars, ±SE. Each group contained 10 mice. From day 15 the differences between the control group and groups treated with CAT2 or CAT1 was significant (P <0.05*). The differences between Control and CAT2 or CAT1 treatment groups were highly significant (P <0.01**) starting at day 22. There is no statistical difference between construct with βdefensin 4 CRD (SEQ ID NO. 9) (low and high dose) and controls.

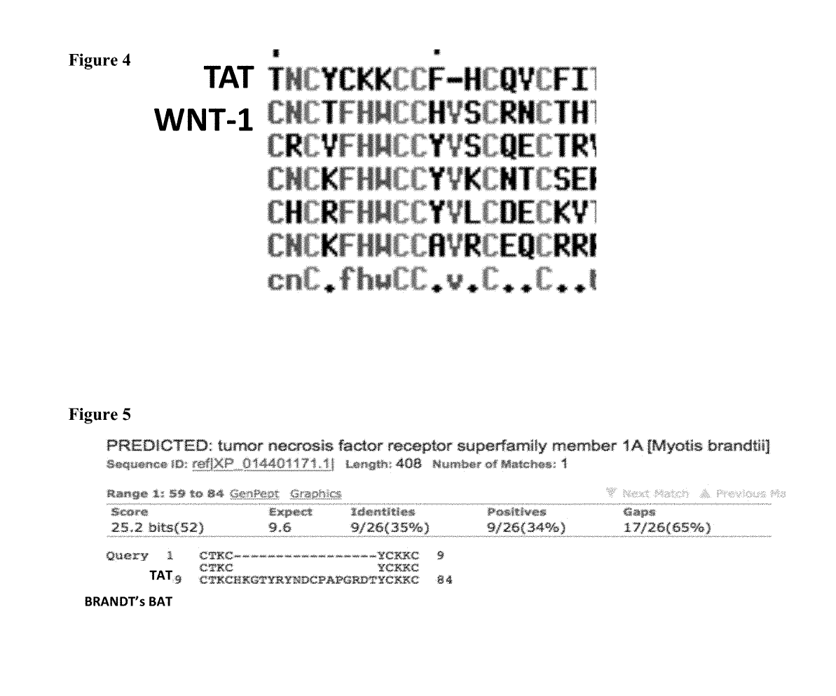

FIG. 4. Homology between TAT CRD and the WNT superfamily of differentiation proteins. (SEQ ID NOS. 10, 11, 11, 12, 13, 14) The alignment is focused toward the COOH side of TAT's CRD.

FIG. 5. Homology between TAT CRD and TNF receptor 1.

The homology is focused into the NH₂ region of TAT's CRD. TAT has completely deleted all intervening sequences between the C-rich stretches of TNFR1 (SEQ ID NO. 15). The identity with bat TNFR1 (SEQ ID NO. 15) is 9/9 amino acids, while the identity with human TNFR1(SEQ ID NO. 15) is 6/9 amino acids, while still completely matched at the two paired C residues. The Human-TAT CRD homology is insufficient to be detected by BlastP, or for that matter by Clustal, and the standard EMBOSS program.

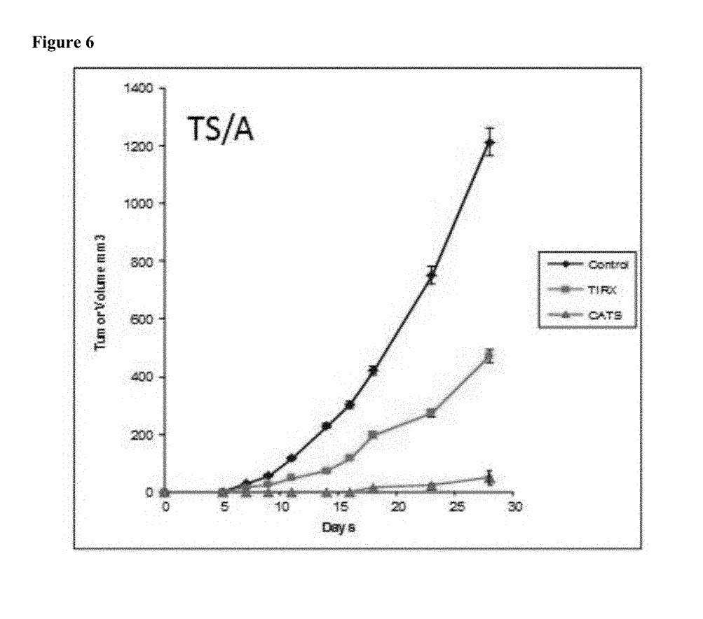

FIG. 6. Comparative antitumor activity of TIRX versus a prototype “humanized” CATS in the TS/A breast cancer model.

A prototype CATs engineered with a modified CRD and an inactivated SH3B has been preliminarily evaluated in the TS/A murine breast cancer model (FIG. 6). Mice orthotopically implanted on Day 0 in the mammary fat pad were administered starting at Day 1 biweekly intravenous injections of 10 ng of inactive control construct (FIG. 6, Control, blue), TIRX (FIG. 6, magenta) or CATS (FIG. 6, orange) and followed for primary tumor mass (FIG. 6) and survival (data not shown). Tumor progressed rapidly in control animals, which were pre-morbid by Day 30. Animals receiving TIRX partially responded to the first four TIRX doses, but after Day 15 further TIRX administration was ineffective since tumor resumed rapid growth (FIG. 6), resulting in death approximately ten days later than controls (not shown). In contrast, animals receiving biweekly CATS exhibited sustained tumor arrest through Day 17 (FIG. 6), after which 4 of 10 animals progressed, albeit at a significantly slower rate, while the remaining six mice remained in remission throughout a five-week course of therapy spanning 10 doses, at which point the trial was terminated because all control mice had died.

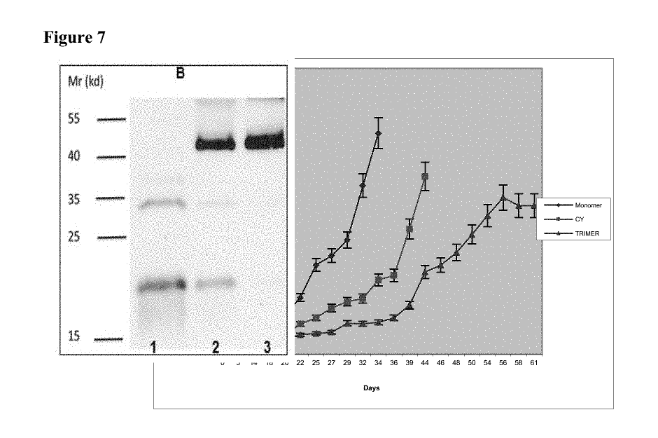

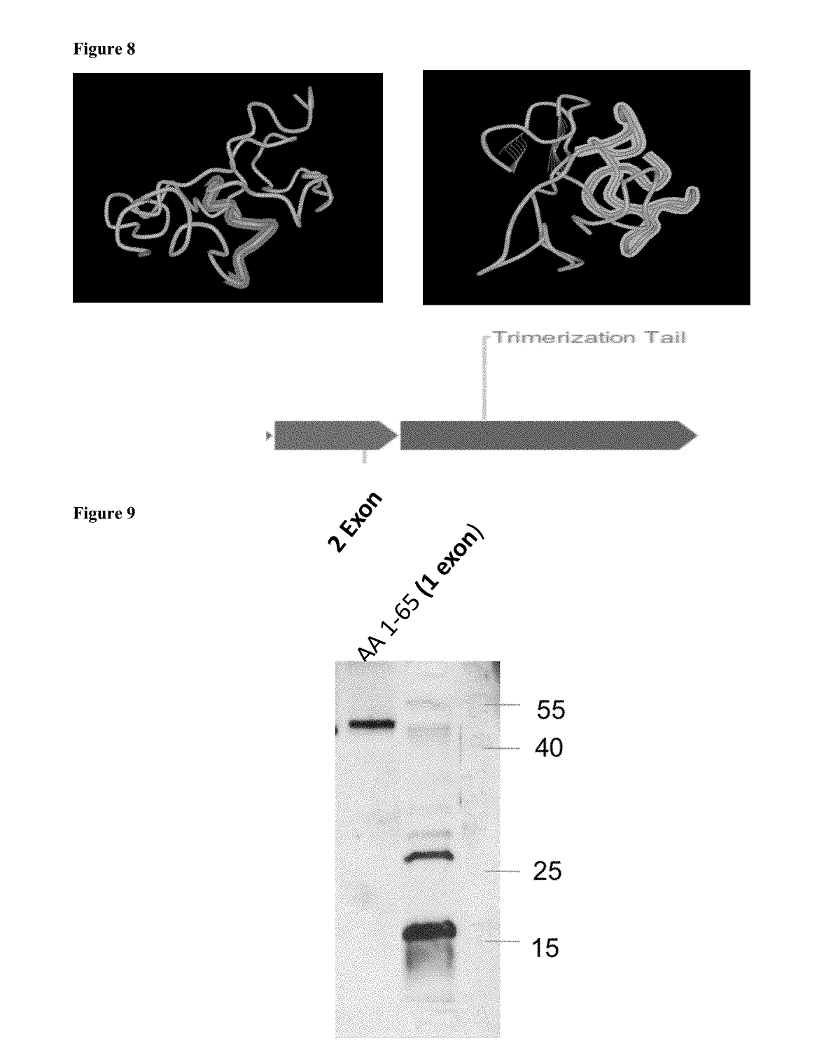

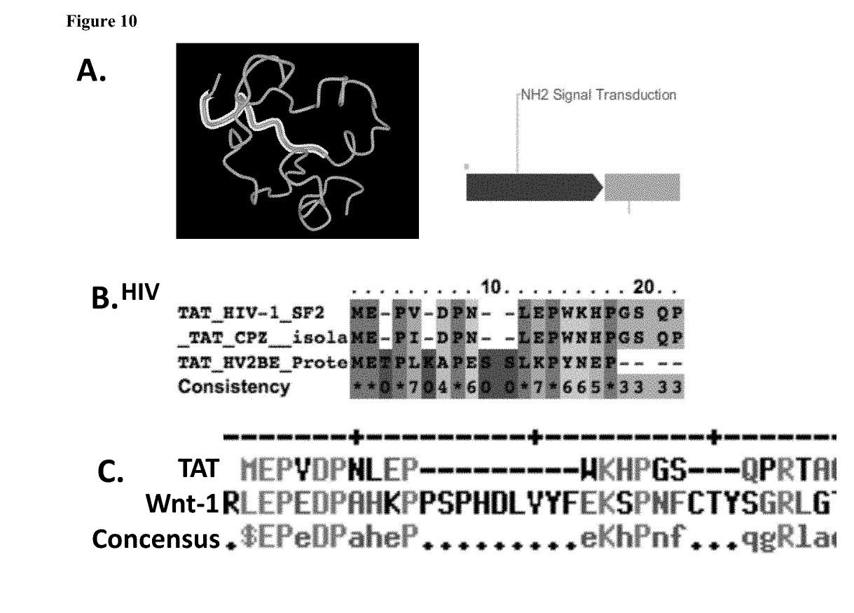

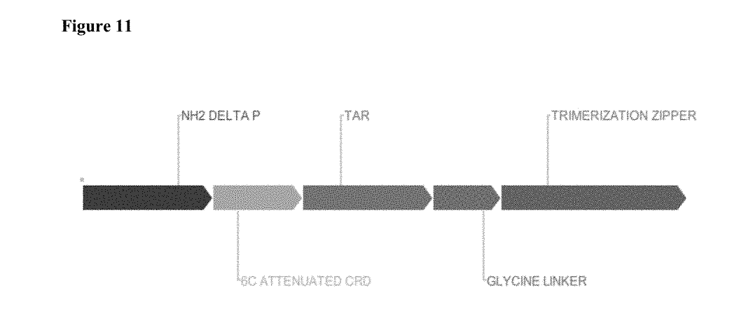

FIG. 7. Graph showing tumor immunomodulatory activity of CATS resident in Trimer.

As seen in FIG. 7, Tat spontaneously forms trimers in baculovirus-infected insect cells. Such cysteine rich multimers possess immunomodulatory activity (Jongrak Kittiworakarn, Alain Lecoq, Gervaise Moine, Robert Thai, Evelyne Lajeunesse, Pascal Drevet, Claude Vidaud, Andre Menez, and Michel Leonetti, HIV-1 Tat Raises an Adjuvant-free Humoral Immune Response Controlled by Its Core Region and Its Ability to Form Cysteine-mediated Oligomers, THE JOURNAL OF BIOLOGICAL CHEMISTRY VOL. 281, 3105-3115, 2006). Stable non-disulfide linked trimeric derivatives such as CRD-containing CATS are immunomodulatory against murine breast cancer in vivo at picomolar dosing (10 ng). At an identical 10 ng dose, monomeric Tat made from the same baculovirus vector after treatment of protein in 10 mM EDTA is inactive as a therapeutic in animals bearing murine breast cancers. At these concentrations monomeric Tat is inactive as well as an extracellular transactivator of the HIV ltr in T cell lines, which typically requires concentrations of 1-10 μg/ml.

FIG. 8. Schematic of the Loop and Tail Regions of Tat.

The linked loop and tail structures of TAT together promote divalent-cation dependent trimerization, and conversion of the trimer to monomer through acid protease cleavage sites (S and T) typically found in the loop approximately at amino acid 65.

FIG. 9. Tail Amino acids (66-101) of TAT mediate spontaneous trimerization in Insect Cells. Tat loops typically contain several acid protease cleavage sites clustered between aa62-65. Cleavage at this site is mimicked by the 1 exon construct. In the cell, acid protease cleavage in the lysosome would be expected to transition trimeric TAT to monomer, and thereby expose TAT's STP among other sequences. FIG. 10. SH3 Binding Domain in Pathogenic HIV-1 TAT has homologies to WNT-1. The alignment of TAT to WNT-1 (SEQ ID NO. 10) in the STP complements the downstream alignment to part of the CRD. Emerging is a pattern where two anchors at either end of an alignment between viral and human genes is interspaced by differing sequences or deletions. Current computer algorithms are ill-equipped to detect these alignments. The dually “anchored ” alignment of TAT and WNT-1 (SEQ ID NO. 10) strengthens the credibility of either.

FIG. 11. Block Depiction of a fully-humanized CATS.

In order is projected an inactivated SH3B with P switched to V, an attenuated CRD with either 6 or 8 C instead of 7 as contained in canonical virulent TATs, TAR/MTS sequence which has no described toxicities, a transition sequence that replaces aa 65-72 in the loop with a non-cleavable poly G linker, and one of several leucine or isoleucine zippers described in this invention that mediate trimerization of E coli lysates.

Although the present invention has been described with reference to specific embodiments, workers skilled in the art will recognize that many variations may be made therefrom and it is to be understood and appreciated that the disclosures in accordance with the invention show only some preferred embodiments and advantages of the invention without departing from the broader scope and spirit of the invention. It is to be understood and appreciated that these discoveries in accordance with this invention are only those which are illustrated of the many additional potential applications that may be envisioned by one of ordinary skill in the art, and thus are not in any way intended to be limiting of the invention. Accordingly, other objects and advantages of the invention will be apparent to those skilled in the art from the detailed description together with the claims.'

| SEQ | | |
|---|---|---|
| ID | | |
| No | Peptide sequence | Source |
| 1 | MEPVDPNLEP WKHPGSQPRT ACTNCYCKKC CFHCQVCFIR KGLGISYGRK | Human |
| | KRRQRRRAPQ DSQTHQASLS KQPASQSRGD PTGPTESKKK VERETETDPF | HIV-1 TAT |
| 2 | MGAMTQLLAG VFLAFLALAT EGGVLKKVIR HKRQSGVNAT LPEENQPVVF | Human tenascin |
| | NHVYNIKLPV GSQCSVDLES ASGEKDLAPP SEPSESFQEH TVDGENQIVF | precursor |
| | THRINIPRRA CGCAAAPDVK ELLSRLEELE NLVSSLREQC TAGAGCCLQP | |
| | ATGRLDTRPF CSGRGNFSTE GCGCVCEPGW KGPNCSEPEC PGNCHLRGRC | |
| | IDGQCICDDG FTGEDCSQLA CPSDCNDQGK CVNGVCICFE GYAGADCSRE | |
| | ICPVPCSEEH GTCVDGLCVC HDGFAGDDCN KPLCLNNCYN RGRCVENECV | |
| | CDEGFTGEDC SELICPNDCF DRGRCINGTC YCEEGFTGED CGKPTCPHAC | |
| | HTQGRCEEGQ CVCDEGFAGV DCSEKRCPAD CHNRGRCVDG RCECDDGFTG | |
| | ADCGELKCPN GCSGHGRCVN GQCVCDEGYT GEDCSQLRCP NDCHSRGRCV | |
| | EGKCVCEQGF KGYDCSDMSC PNDCHQHGRC VNGMCVCDDG YTGEDCRDRQ | |
| | CPRDCSNRGL CVDGQCVCED GFTGPDCAEL SCPNDCHGQG RCVNGQCVCH | |
| | EGFMGKDCKE QRCPSDCHGQ GRCVDGQCIC HEGFTGLDCG QHSCPSDCNN | |
| | LGQCVSGRCI CNEGYSGEDC SEVSPPKDLV VTEVTEETVN LAWDNEMRVT | |
| | EYLVVYTPTH EGGLEMQFRV PGDQTSTIIQ ELEPGVEYFI RVFAILENKK | |
| | SIPVSARVAT YLPAPEGLKF KSIKETSVEV EWDPLDIAFE TWEIIFRNMN | |
| | KEDEGEITKS LRRPETSYRQ TGLAPGQEYE ISLHIVKNNT RGPGLKRVTT | |
| | TRLDAPSQIE VKDVTDTTAL ITWFKPLAEI DGIELTYGIK DVPGDRTTID | |
| | LTEDENQYSI GNLKPDTEYE VSLISRRGDM SSNPAKETFT TGLDAPRNLR | |
| | RVSQTDNSIT LEWRNGKAAI DSYRIKYAPI SGGDHAEVDV PKSQQATTKT | |
| | TLTGLRPGTE YGIGVSAVKE DKESNPATIN AATELDTPKD LQVSETAETS | |
| | LTLLWKTPLA KFDRYRLNYS LPTGQWVGVQ LPRNTTSYVL RGLEPGQEYN | |
| | VLLTAEKGRH KSKPARVKAS TEQAPELENL TVTEVGWDGL RLNWTAADQA | |
| | YEHFIIQVQE ANKVEAARNL TVPGSLRAVD IPGLKAATPY TVSIYGVIQG | |
| | YRTPVLSAEA STGETPNLGE VVVAEVGWDA LKLNWTAPEG AYEYFFIQVQ | |
| | EADTVEAAQN LTVPGGLRST DLPGLKAATH YTITIRGVTQ DFSTTPLSVE | |
| | VLTEEVPDMG NLTVTEVSWD ALRLNWTTPD GTYDQFTIQV QEADQVEEAH | |
| | NLTVPGSLRS MEIPGLRAGT PYTVTLHGEV RGHSTRPLAV EVVTEDLPQL | |
| | GDLAVSEVGW DGLRLNWTAA DNAYEHFVIQ VQEVNKVEAA QNLTLPGSLR | |
| | AVDIPGLEAA TPYRVSIYGV IRGYRTPVLS AEASTAKEPE IGNLNVSDIT | |
| | PESFNLSWMA TDGIFETFTI EIIDSNRLLE TVEYNISGAE RTAHISGLPP | |
| | STDFIVYLSG LAPSIRTKTI SATATTEALP LLENLTISDI NPYGFTVSWM | |
| | ASENAFDSFL VTVVDSGKLL DPQEFTLSGT QRKLELRGLI TGIGYEVMVS | |
| | GFTQGHQTKP LRAEIVTEAE PEVDNLLVSD ATPDGFRLSW TADEGVFDNF | |
| | VLKIRDTKKQ SEPLEITLLA PERTRDITGL REATEYEIEL YGISKGRRSQ | |
| | TVSAIATTAM GSPKEVIFSD ITENSATVSW RAPTAQVESF RITYVPITGG | |
| | TPSMVTVDGT KTQTRLVKLI PGVEYLVSII AMKGFEESEP VSGSFTTALD | |
| | GPSGLVTANI TDSEALARWQ PAIATVDSYV ISYTGEKVPE ITRTVSGNTV | |
| | EYALTDLEPA TEYTLRIFAE KGPQKSSTIT AKFTTDLDSP RDLTATEVQS | |
| | ETALLTWRPP RASVTGYLLV YESVDGTVKE VIVGPDTTSY SLADLSPSTH | |
| | YTAKIQALNG PLRSNMIQTI FTTIGLLYPF PKDCSQAMLN GDTTSGLYTI | |
| | YLNGDKAEAL EVFCDMTSDG GGWIVFLRRK NGRENFYQNW KAYAAGFGDR | |
| | REEFWLGLDN LNKITAQGQY ELRVDLRDHG ETAFAVYDKF SVGDAKTRYK | |
| | LKVEGYSGTA GDSMAYHNGR SFSTFDKDTD SAITNCALSY KGAFWYRNCH | |
| | RVNLMGRYGD NNHSQGVNWF HWKGHEHSIQ FAEMKLRPSN FRNLEGRRKR | |
| 3 | MAMMEVQGGP SLGQTCVLIV IFTVLLQSLC VAVTYVYFTN ELKQMQDKYS | Human |
| | KSGIACFLKE DDSYWDPNDE ESMNSPCWQV KWQLRQLVRK MILRTSEETI | TNF-related |
| | STVQEKQQNI SPLVRERGPQ RVAAHITGTR GRSNTLSSPN SKNEKALGRK | apoptosis- |
| | INSWESSRSG HSFLSNLHLR NGELVIHEKG FYYIYSQTYF RFQEEIKENT | inducing ligand |
| | KNDKQMVQYI YKYTSYPDPI LLMKSARNSC WSKDAEYGLY SIYQGGIFEL | (TRAIL) |
| | KENDRIFVSV TNEHLIDMDH EASFFGAFLV G | |
| 4 | SSGVRLWATR QAMLGQVHEV PEGWLIFVAE QEELYVRVQN GFRKVQLEAR | Human Collagen |
| | TPLPRGTDNE VAALQPP | XVIII Trimer |
| | | Domain |
| 5 | MTLHPSPITC EFLFSTALIS PKMCLSHLEN MPLSHSRTQG AQRSSWKLWL | Saccharomyces |
| | FCSIVMLLFL CSFSWLIFIF LQLETAKEPC MAKFGPLPSK WQMASSEPPC | cerevisiae |
| | VNKVSDWKLE ILQNGLYLIY GQVAPNANYN DVAPFEVRLY KNKDMIQTLT | GCN4 |
| | NKSKIQNVGG TYELHVGDTI DLIFNSEHQV LKNNTYWGII LLANPQFIS | |
| 6 | MTEMSFLSSE VLVGDLMSPF DQSGLGAEES LGLLDDYLEV | Human |
| | AKHFKPHGFS SDKAKAGSSE WLAVDGLVSP SNNSKEDAFS | ATF-4 |
| | GTDWMLEKMD LKEFDLDALL GIDDLETMPD DLLTTLDDTC | |
| | DLFAPLVQET NKQPPQTVNP IGHLPESLTK PDQVAPFTFL | |
| | QPLPLSPGVL SSTPDHSFSL ELGSEVDITE GDRKPDYTAY | |
| | VAMIPQCIKE EDTPSDNDSG ICMSPESYLG SPQHSPSTRG | |
| | SPNRSLPSPG VLCGSARPKP YDPPGEKMVA AKVKGEKLDK | |
| | KLKKMEQNKT AATRYRQKKR AEQEALTGEC KELEKKNEAL | |
| | KERADSLAKE IQYLKDLIEE VRKARGKKRV P | |
| 7 | MTLHPSPITC EFLFSTALIS PKMCLSHLEN MPLSHSRTQG AQRSSWKLWL | Human |
| | FCSIVMLLFL CSFSWLIFIF LQLETAKEPC MAKFGPLPSK WQMASSEPPC | GITRL |
| | VNKVSDWKLE ILQNGLYLIY GQVAPNANYN DVAPFEVRLY KNKDMIQTLT | |
| | NKSKIQNVGG TYELHVGDTI DLIFNSEHQV LKNNTYWGII LLANPQFIS | |
| 8 | MAGNFDSEER SSWYWGRLSR QEAVALLQGQ RHGVFLVRDS | Human |
| | STSPGDYVLS VSENSRVSHY IINSSGPRPP VPPSPAQPPP | CRK |
| | GVSPSRLRIG DQEFDSLPAL LEFYKIHYLD ITTLIEPVSR | |
| | SRQGSGVILR QEEAEYVRAL FDFNGNDEED LPFKKGDILR | |
| | IRDKPEEQWW NAEDSEGKRG MIPVPYVEKY RPASASVSAL | |
| | IGGNQEGSHP QPLGGPEPGP YAQPSVNTPL PNLQNGPIYA | |
| | RVIQKRVPNA YDKTALALEV GELVKVTKIN VSGQWEGECN | |
| | GKRGHFPFTH VRLLDQQNPD EDFS | |
| 9 | MRVLYLLFSF LFIFLMPLPG VFGGIGDPVT CLKSGAICHP VFCPRRYKQI | Human |
| | GTCGLPGTKC CKKP | β-defensin 4 |
| 10 | mglwallpgw vsatlllala alpaalaans sgrwwgivnv asstnlltds | Human |
| | kslqlvleps lqllsrkqrr lirqnpgilh svsgglqsav reckwqfrnr | WNT-1 |
| | rwncptapgp hlfgkivnrg cretafifai tsagvthsva rscsegsies | |
| | ctcdyrrrgp ggpdwhwggc sdnidfgrlf grefvdsgek grdlrflmnl | |
| | hnneagrttv fsemrqeckc hgmsgsctvr tcwmrlptlr avgdvlrdrf | |
| | dgasrvlygn rgsnrasrae llrlepedpa hkppsphdlv yfekspnfct | |
| | ysgrlgtagt agracnsssp aldgcellcc grghrtrtqr vtercnctfh | |
| | wcchvscrnc thtrvlhecl | |
| 11 | maplgyflll cslkqalgsy piwwslavgp qysslgsqpi lcasipgivp | Human |
| | kqlrfcrnyv eimpsvaegi kigiqecqhq frgrrwnctt vhdslaifgp | WNT-3a |
| | vldkatresa fvhaiasagv afavtrscae gtaaicgcss rhqgspgkgw | |
| | kwggcsedie fggmvsrefa darenrpdar samnrhnnea grqaiashmh | |
| | lkckchglsg scevktcwws qpdfraigdf lkdkydsase mvvekhresr | |
| | gwvetlrpry tyfkvpterd lvyyeaspnf cepnpetgsf gtrdrtcnvs | |
| | shgidgcdll ccgrghnara errrekcrcv fhwccyvscq ectrvydvht | |
| | ckpchswatg regrrrwstl gcgprdgclr tghsgpcrsl awiwspgsqg | |
| | hdlleqlprs gglgqcsslq nwtavsgclr dhlgglpggg ehgdts | |
| 12 | CNCKFHWCCY VKCNTCSEI | Human |
| | | WNT-1 (c) |
| 13 | MLEEPRPRPP PSGLAGLLFL ALCSRALSNE ILGLKLPGEP PLTANTVCLT | Human |
| | LSGLSKRQLG LCLRNPDVTA SALQGLHIAV HECQHQLRDQ RWNCSALEGG | WNT-10b |
| | GRLPHHSAIL KRGFRESAFS FSMLAAGVMH AVATACSLGK LVSCGCGWKG | |
| | SGEQDRLRAK LLQLQALSRG KSFPHSLPSP GPGSSPSPGP QDTWEWGGCN | |
| | HDMDFGEKFS RDFLDSREAP RDIQARMRIH NNRVGRQVVT ENLKRKCKCH | |
| | GTSGSCQFKT CWRAAPEFRA VGAALRERLG RAIFIDTHNR NSGAFQPRLR | |
| | PRRLSGELVY FEKSPDFCER DPTMGSPGTR GRACNKTSRL LDGCGSLCCG | |
| | RGHNVLRQTR VERCHCRFHW CCYVLCDECK VTEWVNVCK | |
| 14 | CNCKFHWCCA VRCEQCRRI | Human |
| | | WNT-1 (e) |
| 15 | MMSRSGSGEE DSHTWTYRYN DCPAPGRDTY CKKCENGTYT ASENYLSQCI | TNF receptor 1A |
| | SCSICRKEMG QVEISPCTVD QNTVCGCKKN QYQESLSDTL FRCRNCSPCL | (myotis brandtii) |
| | NGTVQISCSA KQNTVCTCHT GFFLKDNKCV PCDNCEKNTE CTKLCPSTGE | |
| | VIGGSPDSVL LSLVIFFGFC LLCLLFMGLT CHFQRWKPKL QSIGGAGAPA | |
| | LRPRLQPHHR LQSHPQLHAK FHLYPW | |
| 16 | METPLKAPES SLKPYNEPSS CTSERDVTAQ ELAKQGEELL AQLHRPLEPC | HIV-2 |
| | TNKCYCKRCS FHCQLCFSKK GLGISYERKG RRRRTPRKTK TPSPSAPDKS | |
| | ISTRTGDSQP TKRQKKTSEA TVVTTCGLGQ | |
| 17 | MDPIDPDLEP WKHPGSQPRT VCNNCYCKAC CYHCIYCFTK KGLGISYGRK | CPZ TAT |
| | KRTTRRRTAP AGSKNNQDSI PKQPLSQSRG NKEGSEKSTK EVASKTEADQ | |
| 18 | KTACNNCYCK HCSYHCLVCF QKKGLG | 6 Cysteine CRD |
| 19 | KRCTNKCYCK CCCYHCQLCF LQKGLG | 8 Cysteine CRD |

What is claimed is:

1- A humanized immunotherapeutic (CATS) consisting of primary sequence tiles discovered and characterized in this filing, linked together in the same order and functionality as HIV or SIV TAT, and then trimerized to generate an immune therapeutic for treating cancer that is well-tolerated and has minimized autoreactivity.

2- The immunotherapeutic of claim 1 that is any non-natural composition (CATS), as defined by less than 95% homology to any HIV-1 or SIV-1 isolate, with tiles identified in this filing (a, b, d, and e as directly below) linked together in order functioning as:
a. a signal transduction peptide (STP) at an amino terminus;
b. a C-rich determinant (CRD) having homologous human genetic sequences with immunomodulatory ligand activity;
c. a (TAR-MTS);
d. a loop with acid protease cleavage site(s); and
e. a carboxyl trimerization sequence dependent on divalent cation. the resultant trimeric CATS being an immune therapeutic for treating cancer that is humanized so as to reduce its autoreactivity.

3- The humanized trimeric CATS of claim 1 wherein human sequences with identical functionality as the tiles of HIV or SIV Tat are swapped in to replace these sequences with fully human sequences.

4- Claim 3 where human counterpart sequences are identified by a novel algorithm matching anchors in order at the two ends of the functional sequence without giving weight in the match to the intervening sequence.

5- The method of claim 1 where a tile from an SIV Tat lacking pathogenicity is derivatized into the CATS in coordination with other derivatizations.

6- The method of claim 4 where the STP of Tat is replaced by the STP of human wnt-1.

7- The method of claim 4 where the STP of wnt-1 or Tat is modified so as to replace Proline in this SH3 binding domain with Valine.

8- The method of claim 4 where the STP has a WNT-1 like SH3B sequence which is inactivated by replacing all internal Prolines with an amino acid from a group comprising Alanine, Leucine, Isoleucine, Valine, Glycine, and combinations thereof.

9- The method of claim 5 where the STP is derived from SIV Tat so as to activate rather than suppress the immune system.

10- The method of claim 1 where the STP is derived from human CRK and is an activator of the immune system.

11- The method of claim 1 where the immunomodulatory ligand activity is a cysteine-rich ligand for monocyte cells of the innate immune system (CATS).

12- The method of claim 5 where the CRD region is substituted with a natural variant, fully-paired CRD that functions as a ligand-like immunomodulatory for all stages of cancer.

13- The method of claim 5 where the CRD region has 6 Cysteine residues (SEQ ID).

14- The method of claim 5 where the CRD region has 8 Cysteine residues.

15- The method of claim 4 where the CRD contains the CRD from tumor necrosis factor receptor 1 or 2 (TNFR).

16- The method of claim 4 where the CRD contains the CRD from wnt-1.

17- The method of claim 4 where the CRD contains the CRD from TNFR and wnt-1 in tandem.

18- Monoclonal antibodies directed against TNFR used to treat cancer.

19- Monoclonal antibodies directed against wnt-1 used to treat cancer.

20- Bivalent monoclonal antibodies directed against TNFR and wnt-1 used to treat cancer.

21- The method of claim 2 where the divalent-cation dependent trimerization sequence is replaced with an isoleucine or leucine trimerization zipper.

22- The method of claim 21 where the isoleucine zipper is from human TNF-related apoptosis-inducing ligand (TRAIL).

23- The method of claim 21 where the leucine zipper is from human tenascin.

24- The method of claim 21 where the human analogue is ATF-4 (SEQ ID NO.).

25- The method of claim 21 where the trimerization domain is from human collagen XVIII.

26- The method of claim 21 where the trimerization domain is a modified GCN4.

27- The method of claim 21 where each Serine or Threonine residue in the loop region is replaced with an amino acid from a group comprising Alanine, Leucine, Isoleucine, Valine, Glycine, and combinations thereof to provide stabilization.

28- The method of claim 2 where the loop region of Tat is used as a handle or polylinker directly before the trimerization domain to promote trimerization of the whole CATS protein.


SEQUENCE LISTING (19 sequences)

SEQ ID NO: 1 - protein, 100 aa, Homo sapiens
MEPVDPNLEPWKHPGSQPRTACTNCYCKKCCFHCQVCFIRKGLGISYGRKKRRQRRRAPQDSQTHQASLSKQPASQSRGDPTGPTESKKKVERETETDPF

SEQ ID NO: 2 - protein, 2200 aa, Homo sapiens
MGAMTQLLAGVFLAFLALATEGGVLKKVIRHKRQSGVNATLPEENQPVVFNHVYNIKLPVGSQCSVDLESASGEKDLAPPSEPSESFQEHTVDGENQIVFTHRINIPRRACGCAAAPDVKELLSRLEELENLVSSLREQCTAGAGCCLQPATGRLDTRPFCSGRGNFSTEGCGCVCEPGWKGPNCSEPECPGNCHLRGRCIDGQCICDDGFTGEDCSQLACPSDCNDQGKCVNGVCICFEGYAGADCSREICPVPCSEEHGTCVDGLCVCHDGFAGDDCNKPLCLNNCYNRGRCVENECVCDEGFTGEDCSELICPNDCFDRGRCINGTCYCEEGFTGEDCGKPTCPHACHTQGRCEEGQCVCDEGFAGVDCSEKRCPADCHNRGRCVDGRCECDDGFTGADCGELKCPNGCSGHGRCVNGQCVCDEGYTGEDCSQLRCPNDCHSRGRCVEGKCVCEQGFKGYDCSDMSCPNDCHQHGRCVNGMCVCDDGYTGEDCRDRQCPRDCSNRGLCVDGQCVCEDGFTGPDCAELSCPNDCHGQGRCVNGQCVCHEGFMGKDCKEQRCPSDCHGQGRCVDGQCICHEGFTGLDCGQHSCPSDCNNLGQCVSGRCICNEGYSGEDCSEVSPPKDLVVTEVTEETVNLAWDNEMRVTEYLVVYTPTHEGGLEMQFRVPGDQTSTIIQELEPGVEYFIRVFAILENKKSIPVSARVATYLPAPEGLKFKSIKETSVEVEWDPLDIAFETWEIIFRNMNKEDEGEITKSLRRPETSYRQTGLAPGQEYEISLHIVKNNTRGPGLKRVTTTRLDAPSQIEVKDVTDTTALITWFKPLAEIDGIELTYGIKDVPGDRTTIDLTEDENQYSIGNLKPDTEYEVSLISRRGDMSSNPAKETFTTGLDAPRNLRRVSQTDNSITLEWRNGKAAIDSYRIKYAPISGGDHAEVDVPKSQQATTKTTLTGLRPGTEYGIGVSAVKEDKESNPATINAATELDTPKDLQVSETAETSLTLLWKTPLAKFDRYRLNYSLPTGQWVGVQLPRNTTSYVLRGLEPGQEYNVLLTAEKGRHKSKPARVKASTEQAPELENLTVTEVGWDGLRLNWTAADQAYEHFIIQVQEANKVEAARNLTVPGSLRAVDIPGLKAATPYTVSIYGVIQGYRTPVLSAEASTGETPNLGEVVVAEVGWDALKLNWTAPEGAYEYFFIQVQEADTVEAAQNLTVPGGLRSTDLPGLKAATHYTITIRGVTQDFSTTPLSVEVLTEEVPDMGNLTVTEVSWDALRLNWTTPDGTYDQFTIQVQEADQVEEAHNLTVPGSLRSMEIPGLRAGTPYTVTLHGEVRGHSTRPLAVEVVTEDLPQLGDLAVSEVGWDGLRLNWTAADNAYEHFVIQVQEVNKVEAAQNLTLPGSLRAVDIPGLEAATPYRVSIYGVIRGYRTPVLSAEASTAKEPEIGNLNVSDITPESFNLSWMATDGIFETFTIEIIDSNRLLETVEYNISGAERTAHISGLPPSTDFIVYLSGLAPSIRTKTISATATTEALPLLENLTISDINPYGFTVSWMASENAFDSFLVTVVDSGKLLDPQEFTLSGTQRKLELRGLITGIGYEVMVSGFTQGHQTKPLRAEIVTEAEPEVDNLLVSDATPDGFRLSWTADEGVFDNFVLKIRDTKKQSEPLEITLLAPERTRDITGLREATEYEIELYGISKGRRSQTVSAIATTAMGSPKEVIFSDITENSATVSWRAPTAQVESFRITYVPITGGTPSMVTVDGTKTQTRLVKLIPGVEYLVSIIAMKGFEESEPVSGSFTTALDGPSGLVTANITDSEALARWQPAIATVDSYVISYTGEKVPEITRTVSGNTVEYALTDLEPATEYTLRIFAEKGPQKSSTITAKFTTDLDSPRDLTATEVQSETALLTWRPPRASVTGYLLVYESVDGTVKEVIVGPDTTSYSLADLSPSTHYTAKIQALNGPLRSNMIQTIFTTIGLLYPFPKDCSQAMLNGDTTSGLYTIYLNGDKAEALEVFCDMTSDGGGWIVFLRRKNGRENFYQNWKAYAAGFGDRREEFWLGLDNLNKITAQGQYELRVDLRDHGETAFAVYDKFSVGDAKTRYKLKVEGYSGTAGDSMAYHNGRSFSTFDKDTDSAITNCALSYKGAFWYRNCHRVNLMGRYGDNNHSQGVNWFHWKGHEHSIQFAEMKLRPSNFRNLEGRRKR

SEQ ID NO: 3 - protein, 281 aa, Homo sapiens [sequence omitted]

SEQ ID NO: 4 - protein, 67 aa, Homo sapiens
SSGVRLWATRQAMLGQVHEVPEGWLIFVAEQEELYVRVQNGFRKVQLEARTPLPRGTDNEVAALQPP

SEQ ID NO: 5 - protein, 199 aa, Saccharomyces cerevisiae
MTLHPSPITCEFLFSTALISPKMCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 6 - protein, 351 aa, Homo sapiens
MTEMSFLSSEVLVGDLMSPFDQSGLGAEESLGLLDDYLEVAKHFKPHGFSSDKAKAGSSEWLAVDGLVSPSNNSKEDAFSGTDWMLEKMDLKEFDLDALLGIDDLETMPDDLLTTLDDTCDLFAPLVQETNKQPPQTVNPIGHLPESLTKPDQVAPFTFLQPLPLSPGVLSSTPDHSFSLELGSEVDITEGDRKPDYTAYVAMIPQCIKEEDTPSDNDSGICMSPESYLGSPQHSPSTRGSPNRSLPSPGVLCGSARPKPYDPPGEKMVAAKVKGEKLDKKLKKMEQNKTAATRYRQKKRAEQEALTGECKELEKKNEALKERADSLAKEIQYLKDLIEEVRKARGKKRVP

SEQ ID NO: 7 - protein, 199 aa, Homo sapiens; identical to SEQ ID NO: 5

SEQ ID NO: 8 - protein, 304 aa, Homo sapiens
MAGNFDSEERSSWYWGRLSRQEAVALLQGQRHGVFLVRDSSTSPGDYVLSVSENSRVSHYIINSSGPRPPVPPSPAQPPPGVSPSRLRIGDQEFDSLPALLEFYKIHYLDTTTLIEPVSRSRQGSGVILRQEEAEYVRALFDFNGNDEEDLPFKKGDILRIRDKPEEQWWNAEDSEGKRGMIPVPYVEKYRPASASVSALIGGNQEGSHPQPLGGPEPGPYAQPSVNTPLPNLQNGPIYARVIQKRVPNAYDKTALALEVGELVKVTKINVSGQWEGECNGKRGHFPFTHVRLLDQQNPDEDFS

SEQ ID NO: 9 - protein, 64 aa, Homo sapiens
MRVLYLLFSFLFIFLMPLPGVFGGIGDPVTCLKSGAICHPVFCPRRYKQIGTCGLPGTKCCKKP

SEQ ID NO: 10 - protein, 370 aa, Homo sapiens
MGLWALLPGWVSATLLLALAALPAALAANSSGRWWGIVNVASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 11 - protein, 446 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCKPCHSWATGREGRRRWSTLGCGPRDGCLRTGHSGPCRSLAWIWSPGSQGHDLLEQLPRSGGLGQCSSLQNWTAVSGCLRDHLGGLPGGGEHGDTS

SEQ ID NO: 12 - protein, 19 aa, Homo sapiens
CNCKFHWCCYVKCNTCSEI

SEQ ID NO: 13 - protein, 389 aa, Homo sapiens
MLEEPRPRPPPSGLAGLLFLALCSRALSNEILGLKLPGEPPLTANTVCLTLSGLSKRQLGLCLRNPDVTASALQGLHIAVHECQHQLRDQRWNCSALEGGGRLPHHSAILKRGFRESAFSFSMLAAGVMHAVATACSLGKLVSCGCGWKGSGEQDRLRAKLLQLQALSRGKSFPHSLPSPGPGSSPSPGPQDTWEWGGCNHDMDFGEKFSRDFLDSREAPRDIQARMRIHNNRVGRQVVTENLKRKCKCHGTSGSCQFKTCWRAAPEFRAVGAALRERLGRAIFIDTHNRNSGAFQPRLRPRRLSGELVYFEKSPDFCERDPTMGSPGTRGRACNKTSRLLDGCGSLCCGRGHNVLRQTRVERCHCRFHWCCYVLCDECKVTEWVNVCK

SEQ ID NO: 14 - protein, 19 aa, Homo sapiens
CNCKFHWCCAVRCEQCRRI

SEQ ID NO: 15 - protein, 226 aa, Homo sapiens
MMSRSGSGEEDSHTWTYRYNDCPAPGRDTYCKKCENGTYTASENYLSQCISCSICRKEMGQVEISPCTVDQNTVCGCKKNQYQESLSDTLFRCRNCSPCLNGTVQISCSAKQNTVCTCHTGFFLKDNKCVPCDNCEKNTECTKLCPSTGEVIGGSPDSVLLSLVIFFGFCLLCLLFMGLTCHFQRWKPKLQSIGGAGAPALRPRLQPHHRLQSHPQLHAKFHLYPW

SEQ ID NO: 16 - protein, 130 aa, Homo sapiens
METPLKAPESSLKPYNEPSSCTSERDVTAQELAKQGEELLAQLHRPLEPCTNKCYCKRCSFHCQLCFSKKGLGISYERKGRRRRTPRKTKTPSPSAPDKSISTRTGDSQPTKEQKKTSEATVVTTCGLGQ

SEQ ID NO: 17 - protein, 100 aa, Homo sapiens
MDPIDPDLEPWKHPGSQPRTVCNNCYCKACCYHCIYCFTKKGLGISYGRKKRTTRRRTAPAGSKNNQDSIPKQPLSQSRGNKEGSEKSTKEVASKTEADQ

SEQ ID NO: 18 - protein, 26 aa, Homo sapiens
KTACNNCYCKHCSYHCLVCFQKKGLG

SEQ ID NO: 19 - protein, 26 aa, Homo sapiens
KRCTNKCYCKCCCYHCQLCFLQKGLG